(19) United States
(12) Patent Application Publication
(10) Pub. No.: US 2015/0159195 A1
(43) Pub. Date: Jun. 11, 2015

(54) METHOD AND DEVICE FOR RAPID DETECTION OF AMPLIFIED NUCLEOTIDE SEQUENCES

(71) Applicant: CORIS BIOCONCEPT SPRL, Gembloux (BE)

(72) Inventors: Thierry Leclipteux, Wepion (BE); Pascal Mertens, Haltinne (BE); Laetitia Avrain, Etterbeek (BE); Isabelle Ote, Liege (BE); Valerie Smekens, Namur (BE)

(21) Appl. No.: 14/412,600

(22) PCT Filed: Jul. 1, 2013
(86) PCT No.: PCT/EP2013/063777

§ 371 Date: Jan. 2, 2015

(30) Foreign Application Priority Data
Jul. 4, 2012 (BE) 2012/0458

(51) Int. Cl. C12Q 1/68; B01L 3/00; B01L 7/00

(52) U.S. Cl. CPC C12Q 1/686; B01L 7/525; B01L 3/502715; B01L 3/50273; B01L 2300/088; B01L 2300/069; B01L 2300/1805; B01L 2300/1822; B01L 2300/1827; B01L 2400/0481; B01L 2300/0816; B01L 2300/0636; B01L 2200/10

(57) ABSTRACT

A method and a device for fast amplification and detection of target nucleotide sequences possibly present in a sample, combine preferably PCR amplification with oligochromatographic detection by capillarity on a test strip.

### OBJECT OF THE INVENTION

The present invention relates to a method and device for rapid detection of genetic sequences (amplicons) from (genetic) (enzymatic) amplification of an original and specific genetic sequence present in a biological sample.

The method is preferably designed for diagnostic applications on samples comprising different multiple genetic target sequences (oligonucleotides), (i) for fast and efficient detection of specific nucleic sequences in a sample, and correlation of this detection with various pathologies and diseases of an individual from whom stems the tested sample, in particular infectious diseases, bacteremias, respiratory or enteric infectious diseases, including resistant forms to therapeutic agents such as antibiotics, (ii) for identifying contaminations of food products, or (iii) for prognosis tests, notably tests for the possible presence of cancer markers in samples.

### STATE OF THE ART

Among (genetic) amplification methods, Polymerase Chain Reaction (PCR) is the most used technique in molecular biology for obtaining specific amplification of one or of several copies of an original and specific nucleotide sequence possibly present in a biological sample. This technique, by successive (genetic) amplification cycles, allows generation of millions, or even thousands of millions of copies of the original and specific sequence. The PCR amplification method includes repeated thermal cycles for heating and cooling at several (two or three) specific temperatures for denaturating the nucleotide sequences, for hybridization of the primers and enzymatic elongation of the initial nucleotide sequences of hybridized primers.

This method is achieved by means of a programmable instrument which heats and cools a stationary reaction chamber containing the target genetic sequences of the sample to be amplified by the successive thermal cycles, in particular repeated and successive cycles of rises and drops in temperature.

The amplification rate is limited by the heating and cooling rate of the heating blocks of the instrument.

In order to reduce the total time for the (multiple) PCR amplifications, several approaches have been proposed. One of them consists of achieving PCR amplification under a continuous dynamic flow.

With this technique, it is possible to have the sample pass under a constant flow through a (micro)channel which passes through two or three different spatial areas, each having a constant temperature.

Such a device is notably described in the article of Kopp et al. (1998, Science 280, 1046-1048). This device includes a channel passing several times through three areas with different temperatures for denaturation, hybridization and elongation. The sample is introduced at one of the ends of the channel and pumped in a direction towards the other end of the channel where the amplified sample is recovered in order to be analyzed.

The (genetic) amplification in a miniaturized format and in a continuous (constant) dynamic flow also requires detection of the amplified nucleotide sequences.

In the article of Kopp et al., the amplified samples are analyzed by electrophoresis on a gel colored with ethidium bromide outside the amplification device.

As PCR is an extremely sensitive amplification technique, very small amounts of nucleotide sequences (notably from preceding amplifications) capable of contaminating the sample may thus be amplified, generating false positive results. With view to suppressing this drawback, the most efficient approach consists of integrating the (PCR) amplification steps and the steps for detecting the amplified products into a same closed and disposable device (one-use).

Such integrated devices exist for detection, for example by capillary electrophoresis or by hybridization on a micro-checkerboard. However, such methods and devices for measurement and analysis are generally complex and costly. Further, detection on these devices may be perturbed by the emission of micro-bubbles generated during the preliminary (genetic) amplification step.

Therefore, there exists a need for simplification of these methods and devices in order to simplify the use and the efficiency thereof, reduce the cost thereof and increase the rapidity of use thereof.

### AIMS OF THE INVENTION

The present invention aims at providing a method and a device for (genetic) amplification, preferably by PCR in a continuous dynamic flow, combined with a detection method and device which are simple and inexpensive and which do not have the drawbacks of the state of the art.

A particular aim of the present invention is to provide such a method and device which allow rapid detection, but also efficient detection, in order to obtain a result of analysis of the presence of specific nucleic sequences in a sample, preferably a rapid and undistorted diagnostic of the sample, and optionally correlation of this detection with different pathologies and diseases of the individual from whom the sample is collected, in particular, infectious diseases, such as bacteremias, respiratory or enteric infectious diseases including the forms which are resistant to therapeutic agents, in particular antibiotics.

### SUMMARY OF THE INVENTION

A first aspect of the present invention relates to an amplification method, preferably by PCR, and to a detection method of at least one and/or up to several tens (multiplex detection) of target nucleotide sequences possibly present in a biological sample, preferably (i) a sample extracted from an animal or an individual such as a mammal, more particularly a human, or (ii) a contamination of a food composition, said method comprising the following consecutive steps:

a) providing a device comprising:
a channel having a section comprised between about 0.01 mm and about 10 mm,
a test strip in fluidic communication with said channel, said test strip comprising:
either first (one or more) and specific probe(s) complementary to at least one first sequence portion of said amplified target nucleotide sequences or
a capture reagent consisting of a first molecule of a pair of complementary and different molecules, and able to specifically bind to a second (different) molecule of said pair (of different and complementary molecules), this second molecule being bound (directly via a primer which incorporates the amplicon during amplification, preferably PCR, or indirectly via a complementary probe) to said first sequence portion of said amplified target nucleotide sequence(s) and preferably
means for generating at least four (three) areas of preferably different and constant temperatures, three (at least two) of said areas being located at different locations of the channel and the fourth (third) area being located at the test strip.
   These means for generating the three (at least two) areas of preferably different and constant temperatures, are located in different locations of the channel able to generate (genetic) amplification steps, while the fourth (third) area located at the test strip is maintained constant at a value below the three (at least two) other areas, for efficient treatment of the hybridization on the membrane (test strip) in a capillary flow (capillary chromatography).

In the above-described method of the invention, a solution comprising the sample as well as reagents (compounds and optionally additives) for (genetic) amplification are then introduced (according to a step b)) into the channel of the device, in particular of the device of the invention, preferably with PCR, and optionally RT-PCR or MLPA (Multiplex Ligation-dependent Probe Amplification), of the target nucleotide sequence. (According to step c)) amplified and optionally marked sequences of the target nucleotide sequence(s) are thus generated by having the solution circulate according to a dynamic flow (in a fluid), preferably a constant (or continuous) flow in said channel before putting said amplified sequences in contact (according to step d)) with the test strip, and thus generating (according to step e)), by hybridization between the first probes and said amplified sequences (and optionally second marked probes, or markers intercalating DNA), a complex forming a measurable detection signal.

In the method of the invention, the steps b) to e) are generated in the device of step a) designed in a closed way and therefore which cannot (or only very little) be contaminated with other genetic sequences.

According to the method of the invention, the amplified sequences are generated in the channel comprising multiple loops passing over the two (or three) areas of different, constant temperatures and configured for multiple (genetic) amplification cycles, preferably by PCR.

Therefore, in the channel, the multiple loops pass over at least two (preferably two or three) areas of different, constant temperatures and configured for multiple (genetic) amplification cycles, preferably by PCR.

Preferably, in the method of the invention, the solution is introduced (and pushed) into the channel with a syringe pump allowing displacement of the sample in the channel according to a constant dynamic flow. The solution may also be introduced and displaced in a constant dynamic flow in said channel with a suction device. A constant dynamic flow in a closed circuit may also be applied by means of a pump, such as a peristaltic pump (or roller pump).

In the method of the invention, in order to facilitate detection, the reagents for (genetic) amplification, preferably by PCR, are notably pairs of (genetic) amplification primers which may optionally be marked. According to an alternative to this method, it is also possible to achieve marking of the amplified sequence (amplicon) with an intercalating agent of the marked DNA (for example fluorescent marking).

Preferably, at least one of the primers of each pair is labeled.

Preferably, the label of the primer or of the amplified sequence (amplicon) is selected from the group formed by metal particles, in particular gold particles, colloidal particles, polystyrene particles, colored particles, (para)-magnetic particles or fluorescent elements.

According to a preferred embodiment of the method of the invention, the label is a fluorescent label, such as cyanine (Cy5) or a label having similar fluorescent properties.

Advantageously in the method of the invention, the detection signal consists of one or several lines formed by the label, or of one or several dots formed by the label, preferably the signal consists of (a network of) 4, 6, 8, 9, 10, 12, 14 or 16 dots, or even 32 dots or more, formed by the label and therefore present on the surface of the test strip. The selection of the number of dots formed by the label on the surface of the test strip is selected according to the detection resolution and to the facility for obtaining a photograph (image) of the whole of the dots and discrimination between the different dots.

The method of the invention may also put a portion of the non-hybridized sequence of the amplified nucleotide sequences in contact with the first probes, with one or several second probes labelled so as to promote detection of the sandwich type.

The method of the invention allows amplification and detection of any type of target nucleotide sequence regardless of whether it is with a single strand, double strand, or a partly double strand, or a cDNA sequence reverse-transcribed from an RNA sequence.

According to a preferred embodiment of the invention, the method includes an MLPA (Multiplex Ligation-dependent Probe Amplification) step or an RT-PCR step.

In the method of the invention, the readout of the signal is also preferably correlated with the identification of one or several infections, or contamination of a food composition by one or more possibly pathogenic agents, such as genetically modified plants, bacteria or viruses, and/or with identification of the resistance of one or more possibly pathogenic agents to one or several therapeutic treatments, in particular resistance to the action of one or several antibiotics when the possibly pathogenic agent is a bacterium.

The advantage of the method of the invention is its rapidity, since it may be executed rapidly, in less than 90 minutes, preferably in less than 60 minutes, or even under preferred conditions in less than 30 minutes.

The method of the invention also allows amplification and detection of target nucleotide sequences which may be multiple and different, and simultaneously present in the same sample (multiplex conditions). The number of these nucleotide sequences may be 5, 10, 15, 20, 25, 50, 100, 200, 400, 600, 800, 1,000 or more (of different target nucleotide sequences).

The present invention also relates to a device for amplification and (rapid) detection of at least one target nucleotide sequence, comprising means for applying the method of the invention, and comprising at least one channel having a section comprised between about 0.01 mm and 10 mm, a test strip in fluidic communication with said channel, said test strip either comprising first complementary and specific probes of a first sequence portion of the amplified target nucleotide sequences (to be detected) or a capture reagent consisting of at least one first molecule from a pair of different and complementary molecules (such as streptavidin/biotin, avidin/biotin or polystreptavidin/biotin), and able to bind the second molecule of said pair, said second molecule being bound (directly via a primer being incorporated into the amplicon during the amplification, preferably PCR, or indirectly via a complementary probe) to the first sequence portion of the amplified target nucleotide sequences, preferably said channel and said test strip being present in two different chambers.

Advantageously, the device may also comprise means for generating at least four (three) areas with different and constant temperatures, three (at least two) areas being located at different locations of the channel and the fourth (third) area being located at the test strip; said channel may include multiple loops passing over the three (two) areas with different, constant temperatures and configured for multiple (genetic) amplification cycles, preferably by PCR. The device may also comprise at the end of the channel, a syringe pump ensuring the introduction and the preferably constant (or continuous) dynamic flow of the sample within the channel, or comprise other means for ensuring the preferably constant (continuous) dynamic flow of the sample within the channel by suction, or notably a peristaltic pump (roller pump), as well as means for prior treatment of the sample by MLPA or RT-PCR or other prior treatments of the sample.

In the device of the invention, the test strip includes first probes (and optionally second probes (optionally labelled)) able to generate a detection signal in the form of lines or dots on the surface of the test strip after hybridization of the amplified nucleotide sequences. This detection signal may consist (of a network) of 4, 6, 8, 9, 10, 12, 14 or 16 dots, or even 32 dots or more.

Another aspect of the invention relates to a preferably PCR amplification and detection kit, comprising the means of the afore-described device of the invention, as well as the media (reagents) for (genetic) amplification, preferably by PCR, and chromatographic detection on the test strip. These means are preferably probes, preferably labeled, and/or optionally means for diluting samples, in particular a buffer solution, as well as elements customarily used for (genetic) amplification, preferably by PCR, in particular pairs of primers (either single or universal), for which at least one primer of each pair is optionally labeled, as well as one or several internal control sequences for the amplification efficiency (in order to check whether a negative result is a real negative result).

Preferably, as the label is selected from the group consisting of metal particles, in particular gold particles, colloidal particles, colored particles, polystyrene particles, para(magnetic) particles or fluorescent elements, preferably in the kit of the invention, the label is a fluorescent element, more particularly cyanine (Cy5) or a label having similar fluorescent properties.

The present invention will be described in more detail in the preferred embodiment of the invention with reference to the appended figures.

### SHORT DESCRIPTION OF THE FIGURES

FIGS. 1 and 2 schematically illustrate the features of the device of the invention according to a first embodiment. The device comprises a first portion comprising means intended for (genetic) amplification of sequences of nucleic acids in a constant dynamic flow, combined with a second portion comprising means allowing detection of the amplified sequences on a membrane (test strip) by an oligochromatography technique. Both portions are connected through means for transferring amplified sequences, such as a fluidic communication pipe (tube).

FIG. 3 schematically illustrates the features of the device of the invention according to a second embodiment, in which both portions are connected through a channel integrated into the structure of the device.

FIG. 4 schematically illustrates how to place the detection means on the test strip differently in order to generate by hybridization (according to step e)) a complex forming a measurable detection signal.

### DETAILED DESCRIPTION OF THE INVENTION

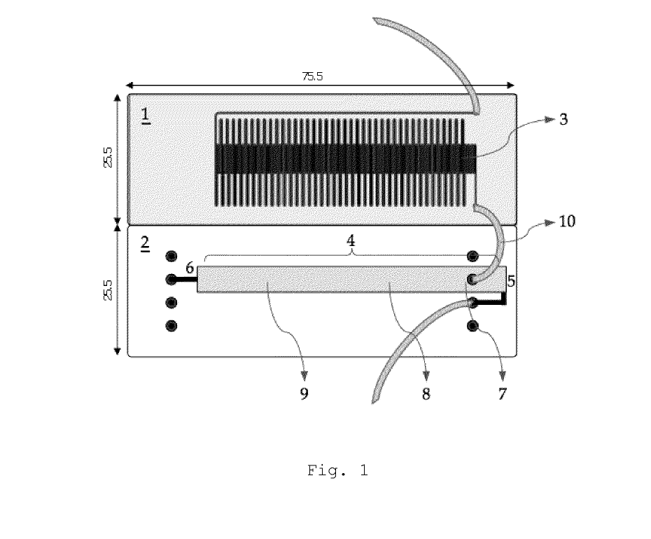

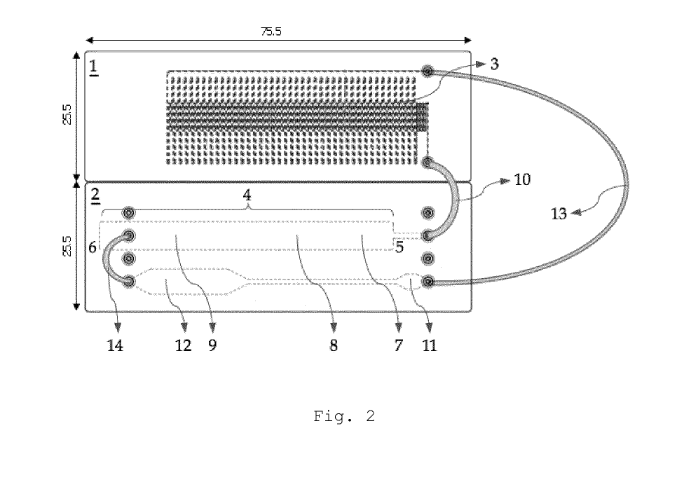

The device as illustrated in FIG. 1 comprises means for microfluidic treatment of a sample allowing rapid and specific (genetic) amplification of sequences (of nucleic acids), in particular PCR type amplification in a constant (or continuous) dynamic flow, combined with means for detecting these amplified nucleotide sequences (amplicons).

As illustrated in FIG. 1, the device of the invention comprises a first (genetic) amplification area or chamber 1 by PCR comprising the different adequate means for carrying out such a (genetic) amplification, and a second area or detection chamber 2 comprising the different adequate means for carrying out such a detection of the amplified genetic sequences from chamber 1.

A device as described in chamber 1 is already known from the state of the art and may include various means for (genetic) amplification, preferably by PCR, in microfluidics and preferably under a constant dynamic flow.

These means comprise a channel 3 made in glass, in quartz or in plastic, in particular in polycarbonate (Bayer Material Science) or in copolymers of cyclo-olefins, and made by injection molding. The microstructural features of this channel are preferably made by a conventional photolithographic treatment.

The aperture of the channel 3 is optionally adapted in the first portion of the device for facilitating introduction and preliminary treatment of the sample to be tested and optionally diluted. Such a sample, preferably a solution comprising the sample, may be integrated into the channel 3 by different means known to one skilled in the art, notably by micro-syringes.

The channel 3 preferably has a coil configuration, the dimensions of which may vary, but which comprises a certain number of loops able to carry out the different steps required for (genetic) amplification, and is put into contact with means for accurately heating areas of these loops. These means may consist in Peltier devices or heating resistors or heating blocks in aluminium and adequate sensors.

The channel 3 preferably has a length comprised between about 1,500 mm and about 2,000 mm, or even more up to 3,000 mm or more, generally of the order of 1,850 mm, a depth of about 100 μm and a width (diameter) of about 200 μm. The coil pattern with multiple loops is provided so as to be able to perform a certain number of (genetic) amplification cycles, preferably more than 15, 20, 25 or 30 or even 50 (genetic) amplification cycles, preferably more than 35 (genetic) amplification cycles, more particularly more than 40 (genetic) amplification cycles, preferably 41 (genetic) amplification cycles in the present example.

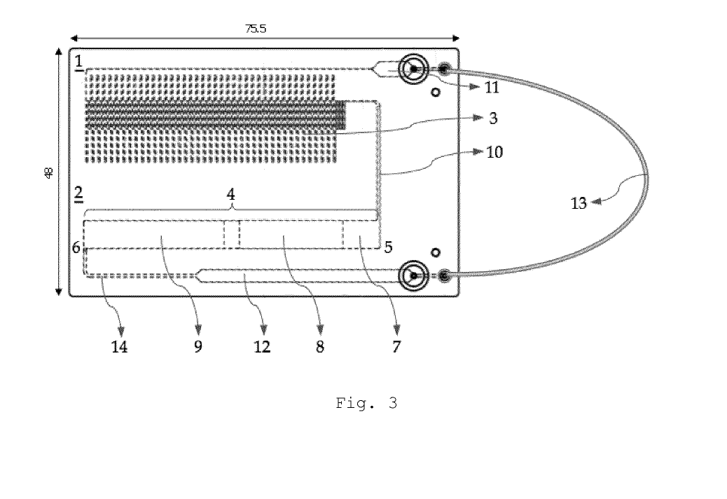

These (genetic) amplification cycles may also be preceded by a pre-denaturation step and followed by a final elongation(post-elongation) step, and the device of the invention may also include one or several reservoirs 11 and 12 giving the possibility of containing the sample to be analyzed and the buffer(s) useful for steps of the method of the invention, such as the oligochromatographic or washing buffer intended to remove the non-specific bonds (hybridizations) of the amplified sequences on the detection support. These reservoirs are preferably filled through apertures of the <<Luer>> type, closed with plugs. Advantageously, the device also includes a pipe 13, preferably a flexible pipe and forming a loop and able to come into contact with a pump head, i.e., with the elements (rollers) of a peristaltic pump (roller pump). This pipe 13 connects, via connectors of the <<olive>> type, the reservoir 11 intended to contain the sample and the reservoir 12 containing the buffer for ensuring fluidic displacement (constant dynamic flow) of the sample and of the buffer, by applying said pump, through the device of the invention. Another connecting means (pipe) 14 also allows connection of the detection means present in the second area or chamber 2 (test strip 4) to the reservoir 12. This closed system as illustrated in FIG. 3 cannot therefore (or only very little) contaminate or be a contaminant.

Preferably, the microfluidic device is made in one piece and the channel 3 is put into contact (via a fluidic communication 10, allowing transfer of the amplified sequences towards the second area or chamber 2) with a detection means present in the second area or chamber 2 of the device of the invention. Preferably, the end of the channel 3 intended for (genetic) amplification joins up (is put into contact) with a test strip 4 allowing detection of the amplified genetic sequences by a method designated as oligochromatography.

Preferably, the means (present on the test strip) intended for detection by oligochromatography are positioned in a second area or chamber 2 separate from the chamber 1 comprising the channel 3. Both chambers are in fluidic communication so that the amplified product (the solution containing the amplified sequences) may be directly transferred into the chamber 2 for its detection in a closed system and preferably which cannot contaminate or be a contaminant.

This second chamber 2 comprises a test strip 4 incorporating three areas or regions. These three areas consist of porous membranes allowing migration by capillarity of a fluid and of its components (i.e., the solution comprising the amplified sequences) from a proximal portion 5 to a distal portion 6. These three areas or regions are an application region 7, a detection region 8 and an absorbing region 9.

Preferably, the membrane of the application area 7 consists of glass fibers or of another adequate material (polyester), the membrane of the detection area 8 consists of nitrocellulose, and the membrane of the absorbing region 9 preferably consists of cellulose. Other types of structure and materials may be contemplated by one skilled in the art for improving the migration characteristics by capillarity within the test strip 4 or for improving the integration of the elements intended for detection on their support. One skilled in the art may for example provide structures of test strips 4 in which the application area 7 is present on the same membrane as the detection area 8 or without any area or absorbing region 9. It is also possible to envision that the test strip 4 is totally covered with nitrocellulose and possibly without any absorbing area.

Different areas or regions may include different reagents able to facilitate the detection of the different amplified genetic sequences put into contact with the test strip 4.

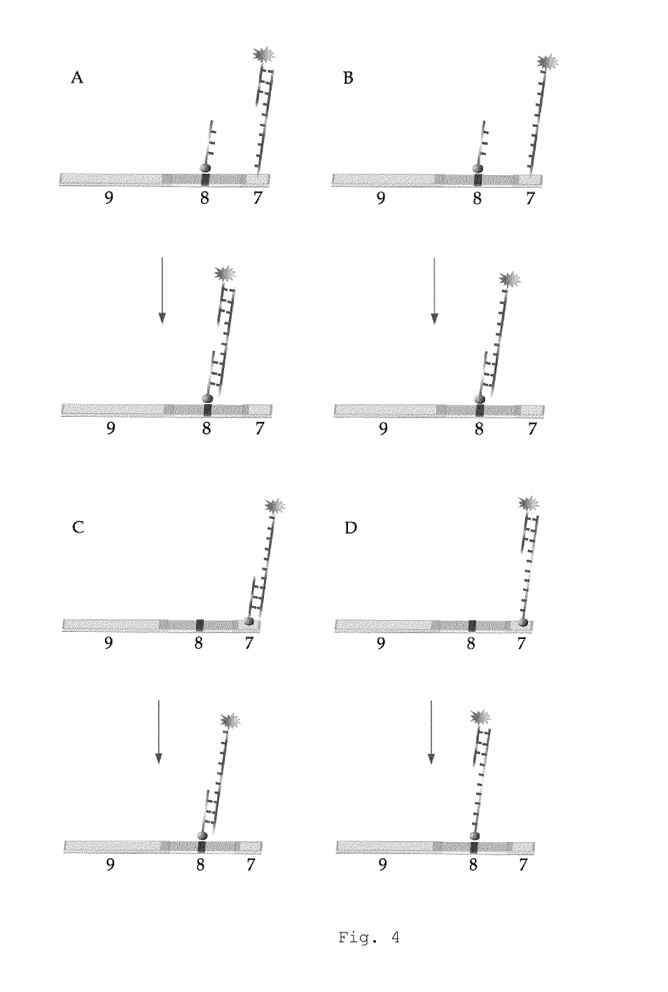

Preferably, the means for putting the (genetic) amplification channel 3 in contact are made on the application area 7 which comprises the specific hybridization means for the amplified genetic sequences (FIG. 4). Advantageously and unexpectedly, this application (via the fluidic communication 10) and the displacement by capillarity on the test strip 4 may be accomplished directly by the flow of the solution comprising the amplified sequences, without requiring the addition of any additional solvent or specific hybridization salts. Preferably, the solution volumes carried away by the channel and deposited on the test strip are comprised between about 10 μl and about 50 μl, preferably between about 30 μl and about 10 μl, preferably of the order of 20 μl.

The representation of the detection may be one or several detection lines, or one or several detection dots (or any other figure, letter, line, dot or figure) present on the membrane of the detection area 8, a detection line or dot being specific to an application of one or several amplified nucleotide sequences from the amplification carried out in the (genetic) amplification channel 3.

Advantageously, the detection reagents are placed on the support of the test strip according to a network in order to generate the adequate figures easily read by a detector, preferably a network with 4, 6, 8, 9, 10, 12, 14 or 16 dots, or even 32 dots or more, able to include at least one or two dots for controlling the detection (at least one dot for controlling amplification (specific hybridization of the specific sequence used as an internal control) and a migration control (detection of the excess of the primer sequences (optionally labelled) not used during the amplification step), as well as positioning dots of the test strip).

The detection means present on the membrane, in particular in the detection area 8, consists of a first capture probe which is hybridized (capture), under specific conditions, to a portion of the specific sequence of one or more amplified nucleotide sequences (FIG. 4).

Preferably, this first capture probe consists of a nucleotide sequence consisting of nucleic acids or of similar molecules (DNA, RNA, PNA, LNA, ANA, HNA, etc.). A carrier reagent may be bound to the first capture probe through a binding element such as a haptene, a peptide or any other molecule which may specifically bind to said carrier reagent.

Preferably, the first capture probe is coupled with a first molecule (first element) of a pair (couple) of different or complementary molecules, such as biotin, itself bound on nitrocellulose via the complementary element of the pair of molecules, such as avidin, streptavidin, polystreptavidin or any other protein binding the biotin, used as a capture reagent.

At the detection, the latter is preferably generated by the excitation (emission) of a fluorescent label which may cause (the emission of) one or several detectable fluorescent signals in the form of lines or dots, or in another geometrical form (figure, drawing) on the surface of the test strip 4. The latter may be viewed via the use of a fluorescence detector. Other means will be selected depending on the type of label used (for example means for measuring the reflectance of probes labeled with gold).

In a first embodiment, the detection will be accomplished in two steps: an amplified target (specific) nucleotide sequence will be hybridized with a complementary and labeled specific probe, present in the area 7 for applying the test strip 4, in order to form a complex between the target sequence and its labeled specific probe, a complex which will migrate on the membrane towards the detection area 8 where it will react with the first capture probe bound in the detection area, thereby forming a complex of the sandwich type. In this complex, a first portion of the amplified target nucleotide sequence is hybridized to a first capture probe allowing immobilization of the target sequence at a specific location of the test strip 4 and a second portion of the amplified target nucleotide sequence is hybridized to a second labeled probe allowing detection of the complex formed and immobilized on the test strip 4 (FIG. 4A). At this instant, the complex will be made visible and detectable by accumulation of the label.

In a second embodiment, the detection will be accomplished in one step: an amplified and labeled target nucleotide sequence (for example by means of a labeled primer which is incorporated into the amplicon during the amplification, preferably PCR) will migrate on the membrane of the test strip 4 towards the detection area 8, where the labeled target sequence will react with the first bound capture probe in the detection area, thereby forming a complex. In this complex, a first portion of the amplified target nucleotide sequence is hybridized to a first capture probe allowing immobilization of the amplified target nucleotide sequence at a specific location of the test strip 4 (FIG. 4B). The presence of a label on the amplified target sequence allows detection of the complex formed and immobilized on the test strip 4.

After having the amplified and labeled target nucleotide sequence migrate towards the detection area 8, it is optionally possible to have a buffered solution migrate, aiming at removing the excess of labeled primers (which have not been incorporated into the amplified sequences) and which may possibly interfere with the measurement of the detection signal. The buffered solution may optionally be introduced (from a reservoir connected to said channel 3) into said channel 3 a few seconds after having introduced the sample into the same channel and the flow of the liquid subject to the amplification and detection steps of the method may be tracked.

Advantageously, the detection means of the device of the invention are by no means perturbed by the introduction or the formation of bubbles in the continuous dynamic flow of the channel, subsequently to thermal variations as this may be the case in detection by capillary electrophoresis or on micro-checkerboards.

According to a preferred embodiment of the invention, the first capture probe is immobilized on the test strip 4 at specific locations prior to executing the test for detecting target nucleotide sequences (FIG. 4). For example, a capture reagent (first molecule of a pair of different and complementary molecules) such as avidin, streptavidin or polystreptavidin, may be bound onto the membrane in the whole detection area 8, and the first capture probe, including the second molecule of the pair of different and complementary molecules, such as biotin, is then deposited on the membrane at specific locations.

The binding is preferably achieved by diluting the capture reagent and the first capture probe in an adequate buffer, and by depositing lines or dots on the membrane, preferably the nitrocellulose membrane. Alternatively, the probe is either bound beforehand on a carrier molecule (such as a pair of complementary molecules (elements) of the biotin-avidin, biotin-streptavidin, anti-biotin antibody and biotin type, . . . ) or on a non-biological support such as a white microsphere (not visible with the detection system) before deposition, or directly (chemically) bound onto the membrane of nitrocellulose.

In the case of deposition of lines, the distribution rate of the capture reagents may vary between about 50 mm and about 10 mm per second, but is preferably set to a value of about 30 mm per second.

The volume of distributed materiel may vary between 0.5 μl/cm and about 3 μl/cm, but preferably is of the order of about 1 μl/cm.

In the case of deposition of dots, the volume of distributed materiel may vary between 100 nl and 1 nl, but is preferably set to a value of about 10 nl.

The concentration of the capture reagents may vary between about 0.01 mg/ml (or a lower concentration) or about 0.1 mg/ml and about 5 mg/ml, preferably of the order of 1.5 mg/ml.

The concentration of the first capture probes may vary between about 50 μM and about 1 μM or about 1 nM or a lower concentration, preferably between about 30 μM and about 5 μM, more particularly of the order of 10 μM.

According to a preferred embodiment of the invention, the buffer used for the binding consists of a saline solution (NaCl) buffered with a phosphate at a pH of about 7.2.

When the support consists of a nitrocellulose membrane, the test strip 4 is then dried for a few seconds or minutes (two minutes or more) up to about 72 hours at a temperature of about 50° C., or at a temperature of about 60° C. or more.

According to a preferred embodiment of the invention, the oligochromatographic test strip 4 used in the device of the invention has a size of about 5 mm in width for a length of about 57 mm, but other shapes and formats of test strips may be used.

In the device and method of the invention, the amplified sequences which may be detected, may be DNA sequences, in particular single-strand or double-strand or partially double-strand DNA sequences, as well as cDNA sequences obtained by reverse transcription of RNA sequences.

The reverse transcription step may optionally be integrated into the method of the invention within a separate unit of the device of the invention, for example in a chamber or a (micro)channel connected (optionally through valves or pumps) to the channel 3 where (genetic) amplification occurs, preferably by PCR.

The method and the device of the invention are perfectly adapted for amplification and detection of multiple and different nucleotide sequences present in the same sample (multiplex conditions).

The number of different nucleotide sequences which may be detected in the device of the invention, may be adapted according to the needs required by the application. The method and device of the invention may be modulated and allow adaptation of the number of probes present on the test strip according to the number of target nucleotide sequences to be detected.

The number of (specific and/or universal) primer pairs will have to be adapted to the number of target nucleotide sequences to be amplified.

In a preferred embodiment, between about 2 sequences and about 50 target nucleotide sequences, preferably between about 5 sequences and about 20 target nucleotide sequences, are amplified and detected in the same sample.

Beyond a certain number of specific primer pairs present in the same amplification solution, the efficiency of the (PCR) amplification decreases.

In order to increase the number of target nucleotide sequences which may be detected in a same sample, it is possible to use the MLPA (Multiplex Ligation-dependent Probe Amplification) technique. MLPA is an alternative of multiplex PCR and allows amplification of multiple target nucleotide sequences with a pair of universal primers. The amplification, preferably by PCR, is preceded by a ligation step. Each nucleotide sequence to be amplified is hybridized to a pair of oligonucleotides recognizing two sites (parts or portions) adjacent to the targeted nucleotide sequence. Each oligonucleotide of the pair further includes a universal sequence which is complementary to one of the primers. Thus, once the pairs of oligonucleotides have been hybridized to their target nucleotide sequence, they may be ligated together by means of a ligase. The ligated oligonucleotides are then amplified by a pair of universal primers.

The ligation step may optionally be integrated into the method of the invention and carried out within a unit separate from the device of the invention, for example in a chamber or (micro)channel connected to the channel 3 where (genetic) amplification occurs, preferably by PCR, with a pair of universal primers.

In an embodiment using MLPA, between 20 sequences and 1000 target nucleotide sequences are amplified and detected in the same sample.

The sample is preferably an aqueous solution containing purified DNA as well as other components which may be used in the amplification device under a constant dynamic flow.

These different compounds are preferably introduced into the channel 3 at the same time as the samples or optionally through other (micro)channels or reservoirs put into contact with the channel 3.

These other components are reagents able to carry out this (genetic) amplification, preferably (genetic) amplification by PCR, notably a DNA polymerase, able to obtain polymerization of the DNA in an adequate buffer, optionally supplemented with the addition of other compounds (reagents) such as magnesium chloride, primers, in particular pairs of adequate primers (either specific or universal) for (genetic) amplification, dNTPs selected according to the required type of (genetic) amplification, preferably by PCR, and to the type of genetic sequence(s) to be detected.

Other components such as surface blocking (saturating) agents, preferably serum albumin (bovine serum albumin, BSA) and polyethylene glycol (PEG) as blocking agents, or stabilizers, as well as reaction enhancers, may also be added to the reaction solution.

The selection of nucleotide sequences used as primers (either specific or universal) for (genetic) amplification, preferably by PCR, is made according to the type of nucleotide sequence(s) to be amplified and to be detected. The selection of these primers is within the reach of one skilled in the art as well as their optional labeling with elements such as metal particles, preferably gold particles, colloids or fluorescent elements.

Preferably, the label is a fluorescent label, preferably consisting of cyanine (Cy5) or of a label having similar fluorescent properties.

The labeling may also be indirect, via contacting of the non-labeled amplified sequences with second labeled probes complementary to a part (or portion) of the amplified sequence non-hybridized to the first capture probes (sandwich detection).

The labels having different types of coloration may also be used for labeling different primer sequences so as to obtain amplification of the DNA sequences in a multiplex form and so as to facilitate their simultaneous or consecutive detection in the test strip 4.

Each color is specific to a sequence or a group of amplified genetic sequences and initially present in the tested sample, and their colorimetric detection may be obtained by means known to one skilled in the art.

The device of the invention also includes the elements capable of measuring the signal, in particular a fluorescent signal consisting of one or several dots or lines formed by the label (fluorescent) on the test strip 4, notably a signal detector, in particular a fluorescent signal detector.

Preferably, a light source generates a light beam in order to excite the fluorescence of the label. The detection has to be adjusted so as to obtain the same detection efficiency on the surface of the test strip 4 comprising the whole of the dots or lines to be analyzed. A detector used in this context is a CCD camera capable of taking a photograph of the whole of the dots or lines (or of any other figure, drawing, letter or numerical figure).

In addition to the detection unit, the device of the invention includes other elements able to facilitate this (genetic) amplification, preferably by PCR, and this detection, notably a heating unit (for example a heating resistor or aluminium blocks) of the lower surface of the microfluidic device, this heating device being designed so as to obtain a (genetic) amplification reaction, preferably by PCR, and detection by heat treatment of the adequate portions of the microfluidic device.

For example, the chambers 1 and 2 of the device of the invention rest on four (at least three) heating blocks, for which the temperature is controlled independently of each other. Three (at least two) blocks are used for regulating the temperature of the (genetic) amplification, preferably by PCR, and are located below the chamber 1 in different areas of the channel 3, and the fourth (third) block is located below the chamber 2 at the test strip 4.

In order that the (genetic) amplification preferably by PCR be carried out with a high yield, it is important that the transition from one temperature area to another be well defined. For this purpose, the heating blocks are separated from each other by a spacing from about 1 mm to about 2 mm.

The heating unit may also include a lid or any other device in order to limit heat losses.

The channel 3 includes different portions brought to (treated at) different temperatures so as to facilitate (genetic) amplification, preferably by PCR, while the detection chamber 2 is maintained at a single adequate temperature.

Preferably, the temperatures may vary between about 100° C. and about 90° C. (preferably about 98° C.), and about 50° C. and about 80° C. (preferably about 57° C.) in the amplification area or chamber 1, while the detection area or chamber 2 is maintained at a temperature comprised between about 40° C. and about 60° C. (preferably about 41° C.)

The detection unit and the heating unit may be integrated into a same device, or on the contrary separated according to the needs of the user. Given that (genetic) amplification, preferably by PCR, requires a longer time than the measurement of the signal, it may be advantageous to separate both units.

The different chambers or areas 1 and 2 are connected by means of adequate elements, notably standard piping systems (tubing), preferably in polyethylene or in silicone, or via a pumping system preferably including means such as pumps, syringes, valves, etc. A syringe pump may for example be used for ensuring pumping of the sample (at a constant and controlled temperature) so as to also allow a constant flow through the channel 3.

This syringe-pump system will preferably include an internal diameter comprised between about 6 mm and about 30 mm, a pumping rate comprised between about 0.012 mm and about 6 mm per second, for a minimum pumping volume of the order of 0.3 μl per minute.

A constant flow inside the device may also be ensured by a peristaltic pump (roller pump) in a closed circuit, but for which the rate may be variable.

The method of the invention is carried out in a very rapid way in less than 90 minutes, preferably in less than 60 minutes and even in less than 30 minutes. The (genetic) amplification, preferably by PCR, is carried out in less than 60 minutes and preferably in less than 30 min, and the detection on the test strip in less than 15 minutes, preferably in less than 5 minutes (including detection and development).

1-36. (canceled)

37. A method for PCR amplification and for detecting at least one target nucleotide sequence, said method comprising the following consecutive steps:
a) providing a device comprising:
a channel having a section comprised between about 0.01 mm and about 10 mm;
a test strip in fluidic communication with the channel, said test strip comprising:
either one or more first complementary and specific probes to at least one first sequence portion of at least one amplified target nucleotide sequence; or
at least one first molecule of a pair of different and complementary molecules, able to bind a second molecule from said pair, said second molecule being bound to the first sequence portion of the at least one amplified target nucleotide sequences;
at least four areas of different and constant temperatures, at least two or three areas being located at different locations of the channel and the fourth area being located at the test strip;
b) introducing into the channel a solution comprising the sample and the amplification reagents for the at least one target nucleotide sequence;
c) generating amplified sequences of the at least one target nucleotide sequences by having the solution circulate according to a constant dynamic flow in the channel;
d) putting said at least one amplified sequence in contact with the test strip; and
e) generating by hybridization between the first probes and said at least one amplified sequence, a complex forming a measurable detection signal.

38. The method of claim 37, wherein the steps b) to e) are generated in the device of step a) designed in a closed configuration.

39. The method of claim 37, wherein the amplified sequences are generated in the channel, said channel comprising multiple loops passing over two or three areas of different, constant temperatures and configured for multiple amplification cycles.

40. The method of claim 37, wherein the solution is introduced into the channel with a syringe-pump allowing displacement of the sample in the channel according to a constant dynamic flow.

41. The method of claim 37, wherein the solution is introduced and displaced in a constant dynamic flow in the channel by a suction device

42. The method of claim 37, wherein the solution is displaced in a constant dynamic flow by a peristaltic pump.

43. The method of claim 37, wherein the step of generating amplified sequences is obtained by adding one or more pairs of primers and wherein at least one of the primers of each pair is labeled.

44. The method of claim 43, wherein the primer is labelled with a label selected from the group formed by metal particles, colloidal particles, colored or paramagnetic particles, polystyrene particles or fluorescent elements.

45. The method of claim 44, wherein the fluorescent label is cyanine (Cy5).

46. The method of claim 37, wherein the detection signal consists of one or more lines or dots formed by the label.

47. The method of claim 37, wherein the detection signal consists of 4, 6, 8, 9, 10, 12, 14, 16 or 32 dots formed by the label.

48. The method according of claim 37, wherein a portion of the sequence of the at least one amplified nucleotide sequence is non-hybridized and is put into contact with the first probes, with one or more second labeled probes.

49. The method of claim 37, wherein the target nucleotide sequence is a single strand, double strand, partly double strand sequence or a reverse-transcribed cDNA sequence from an RNA sequence.

50. The method of claim 37, which further comprises, prior to and including step b), a Multiplex Ligation-dependent Probe Amplification (MLPA) reaction.

51. The method of claim 37, wherein signal readout is correlated with the identification of one or more infections by one or more pathogenic agents, and/or with the identification of the resistance of one or more pathogenic agents to one or more therapeutic treatments

52. The method of claim 51, wherein the pathogenic agent is a bacterium and the resistance is resistance to one or more antibiotics.

53. The method of claim 37, wherein signal readout is correlated to identification of food product contamination.

54. The method of claim 37, the method being adapted for amplifying and detecting multiple and different nucleotide sequences present in a same sample.

55. The method of claim 37, wherein the pair of different and complementary molecules is selected from the group consisting of streptavidin/biotin, avidin/biotin, streptavidin/biotin or an anti-biotin/biotin antibody.

56. A device for rapid PCR amplification and detection of at least one target nucleotide sequence present in a sample, said device comprising:
a channel having a section comprised between about 0.01 mm and about 10 mm;
a test strip in fluidic communication with said channel, said test strip comprising either first complementary and specific probes of a first sequence portion of the at least one amplified target nucleotide sequence, or at least one first molecule of a pair of different and complementary molecules, and able to bind a second molecule of said pair, said second molecule being bound to the first sequence portion of the at least one amplified target nucleotide sequence; and
wherein the channel comprises multiple loops passing over at least two or three areas of different, constant temperatures and configured for multiple genetic amplification cycles.

57. The device of claim 56 comprising at least four areas of different and constant temperatures, three areas being located at different locations of the channel and the fourth area being located at the test strip.

58. The device of claim 56, comprising on a channel end, a syringe-pump ensuring introduction and constant dynamic flow of the sample within the channel.

59. The device of claim 56, wherein the test strip includes first probes and optionally second probes able to generate a detection signal in the form of lines or dots on a test strip surface after hybridization of the at least one amplified nucleotide sequence.

60. The device of claim 56, wherein the different and complementary molecules pair is selected from the group consisting of streptavidin/biotin, avidin/biotin, polystreptavidin/biotin or an anti-biotin/biotin antibody.

61. A PCR amplification and detection kit for at least one target nucleotide sequence present into a sample, said kit comprising the device of claim 56, and media and reagents for genetic amplification by PCR and oligochromatographic detection on a test strip.

62. The kit of claim 61, comprising labeled primers, labeled probes and/or means for diluting the sample.

63. The kit of claim 62, wherein a label of labeled primers and labeled probes is selected from the group consisting of metal particles, polystyrene particles, colored or magnetic particles, colloidal particles or fluorescent elements.

64. The kit of claim 63, wherein the fluorescent element is cyanine (Cy5).
